(19) United States
(12) Patent Application Publication
(10) Pub. No.: US 2009/0105767 A1
(43) Pub. Date: Apr. 23, 2009

(54) Total joint subsidence protector

(75) Inventor: Mark A. Reiley, Piedmont, CA (US)

Correspondence Address: DUANE MORRIS LLP - Philadelphia;IP DEPARTMENT, 30 SOUTH 17TH STREET, PHILADELPHIA, PA, 19103-4196, US

(73) Assignee: InBone Technologies, Inc.

(21) Appl. No.: 11/975,185

(22) Filed: Oct. 18, 2007

(51) Int. Cl. A61B 17/56

(52) U.S. Cl. 606/301

(57) ABSTRACT

A subsidence protection device is provided. The subsidence protection device may be placed in either the tibia or the talus to support a portion of a total ankle prosthesis.

### BACKGROUND OF THE INVENTION

On occasion either the caudal or cephalad part of a total joint prosthetic can subside into the bone. For example, the talar component of a total ankle replacement can subside into the talus. This subsidence can cause pain to the patient. It is therefore desired to provide a device that protects a total joint replacement prosthesis from subsidence.

### SUMMARY OF THE INVENTION

The present invention provides a device for preventing subsidence of a portion of a total joint prosthetic.

The present invention may include an elongate body with a head portion.

The body may take on various shapes and have various cross sections. The body may be made of a prosthetic material. The body may be made of a biological material. The body may be covered with a bony in-growth surface.

The body may be inserted into the talus to prevent the caudal portion of a prosthesis from subsiding.

The body may be inserted into the tibia to prevent the cephalad portion of a prosthesis from subsiding.

Other objects, advantages, and embodiments of the invention are set forth in part in the description which follows, and in part, will be obvious from this description, or may be learned from the practice of the invention.

### BRIEF DESCRIPTION OF THE DRAWINGS

FIG. 1 is a lateral view of a human lower leg and foot skeleton.

FIG. 2 is a lateral view of the human lower leg and foot skeleton of FIG. 1 with the fibula 12 shown in an assembly format and having a planarly resected tibia and talus.

FIG. 3 is a lateral view of the lower leg and foot showing a lower prosthetic body which has subsided into the talus.

FIG. 4 is a lateral view of the lower leg and foot showing an upper prosthetic body which has subsided into the tibia.

FIG. 5 is a perspective view of a treatment device according to the present invention.

FIG. 6a is a lateral view of the lower leg and foot showing the treatment device of FIG. 5 inserted into the talus.

FIG. 6b is a lateral view of the lower leg and foot showing the treatment device of FIG. 5 inserted through the talus and into the calcaneous.

FIG. 7 is a lateral view of the lower leg and foot showing the treatment device of FIG. 5 inserted into the tibia.

FIG. 8a is a lateral view of the lower leg and foot illustrating a bore formed in the talus remaining after withdrawal of the drill bit.

FIG. 8b is a schematic similar to FIG. 8a and illustrating insertion of a treatment device into the pre-formed bore in the talus.

FIG. 9a is a lateral view of the lower leg and foot illustrating a bore formed in the tibia remaining after withdrawal of the drill bit.

FIG. 9b is a schematic similar to FIG. 9a and illustrating insertion of a treatment device into the pre-formed bore in the tibia.

FIG. 10 is a perspective view of an alternate embodiment of a treatment device according to the present invention.

FIG. 11 is a perspective view of an alternate embodiment of a treatment device according to the present invention.

FIG. 12 is a perspective view of an alternate embodiment of a treatment device according to the present invention.

FIG. 13 is a perspective view of an alternate embodiment of a treatment device according to the present invention.

FIG. 14 is a perspective view of an alternate embodiment of a treatment device according to the present invention.

FIG. 15 is a perspective view of an alternate embodiment of a treatment device according to the present invention.

FIG. 16 is a perspective view of an alternate embodiment of a treatment device according to the present invention.

FIG. 17 is a perspective view of an alternate embodiment of a treatment device according to the present invention.

FIG. 18 is a perspective view of an alternate embodiment of a treatment device according to the present invention.

FIG. 19a is a lateral view of the lower leg and foot illustrating a pilot hole formed in the talus remaining after withdrawal of the drill bit.

FIG. 19b is a schematic similar to FIG. 19a and illustrating a saw forming a bore in the talus.

FIG. 19c is a schematic similar to FIG. 19b and illustrating insertion of a treatment device into the pre-formed bore in the talus.

### DESCRIPTION OF THE PREFERRED EMBODIMENT

### I. Anatomy of the Ankle

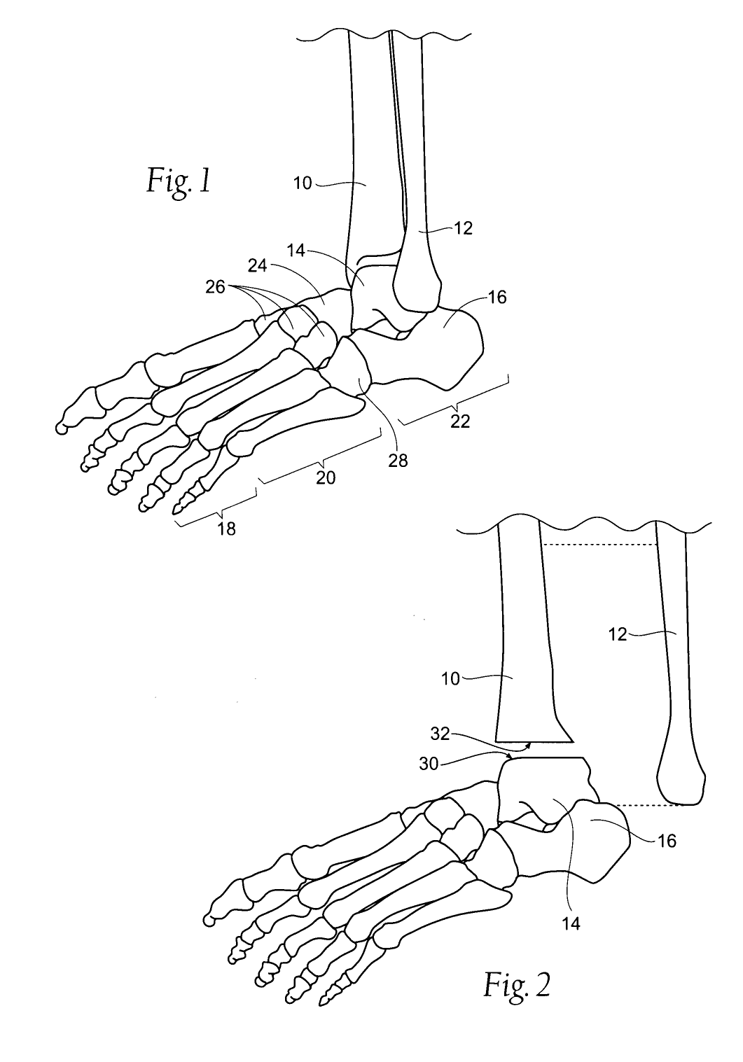

Referring to FIG. 1, the lower leg comprises the tibia 10 and the fibula 12. The tibia 10 and the fibula 12, along with the talus form the ankle joint which allows for the up and down movement of the foot. The subtalar joint, located below the ankle is made of the talus 14 and calcaneous 16. The subtalar joint allows for side to side movement of the foot.

FIG. 1 further shows the foot, which comprises fourteen phalanges or toe bones 18 connected to the metatarsus bones 20. There are also seven tarsal bones 22, of which the talus 14 supports the tibia 10 and the fibula 12, and the heel bone or calcaneous 16. Of the tarsal bones, the talus 14 and the calcaneous 16 are the largest and are adjacent to each other. The other tarsal bones include the navicular 24, three cuneiforms 26, and the cuboid 28.

FIG. 2 shows a lower leg in which the tibia 10 and talus 14 have been resected, leaving two planar portions 30,32. It is desirable to cut away the inferior end of the tibia 10 to leave a tibial planar surface 32 and/or the superior end of the talus 14 to form talar planar surface 30 when performing a total ankle replacement.

### II. Ubsidence

In patients with a total ankle joint replacement, it is possible for a portion of the prosthesis to subside into the bone. FIGS. 3 and 4 show lower legs with total ankle joint replacements. Either the caudal portion 34 (FIG. 3) of the prosthesis may subside into the talus 14 or the cephalad portion 36 (FIG. 4) of the prosthesis may subside into the tibia 10. Subsidence of the prosthesis can cause pain to the patient. If a portion of a prosthesis subsides, the forces in the joint can become unbalanced and may cause pain to the patient. In some cases subsidence may become such a problem that an additional surgery is required to either remove or replace the joint replacement.

### III. Ubsidence Protector

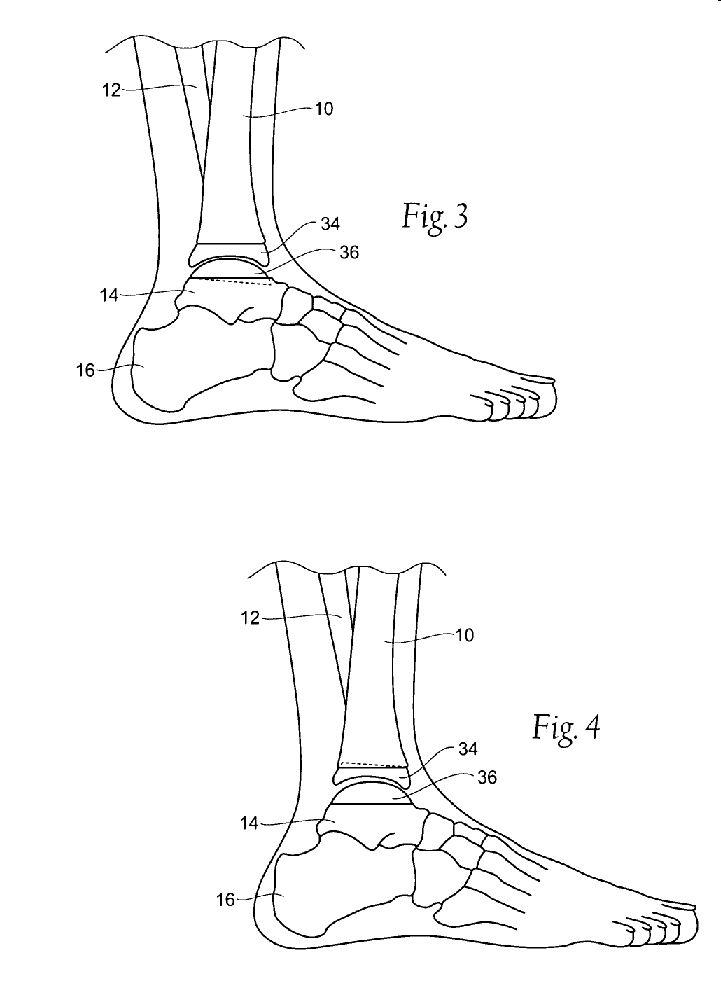

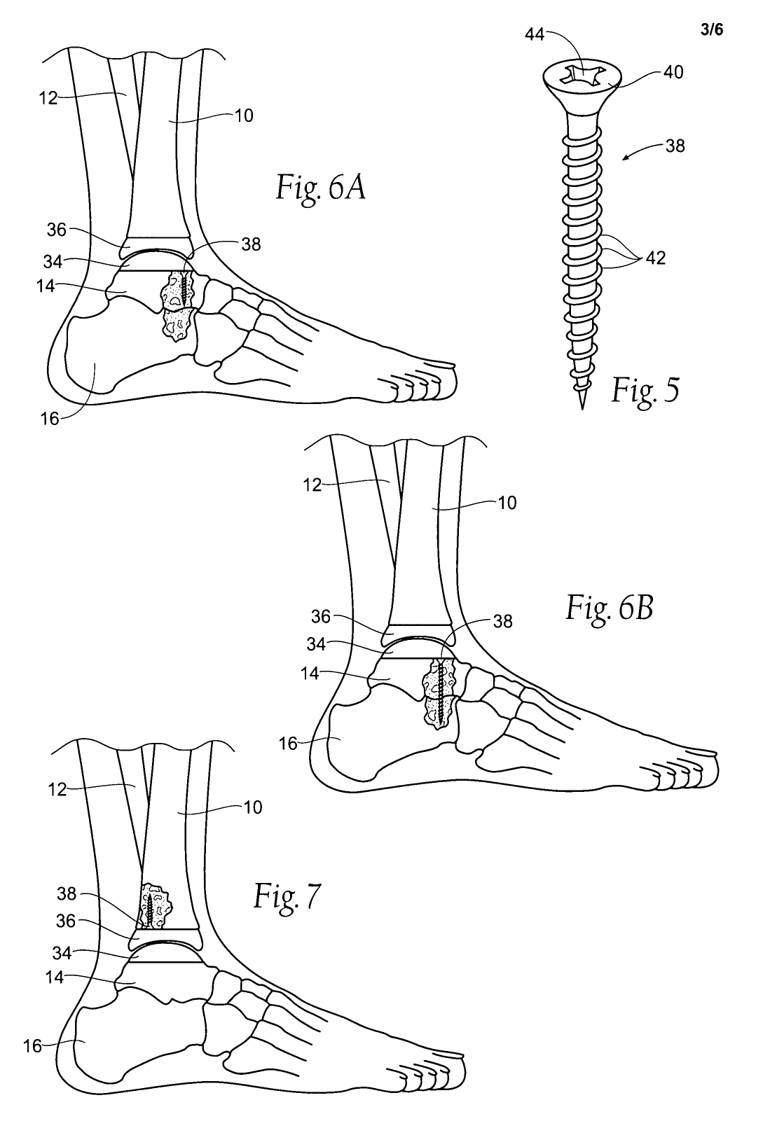

FIG. 5 shows a device 38 for protecting subsidence according to the present invention. This subsidence protection device 38 may be inserted into the bone beneath the prosthesis in the area or areas where the prosthesis is most likely to subside. For example, as shown in FIG. 6, the device 38 may be inserted into the talus 14 just under the anterior lip of the caudal prosthesis component 34. Likewise, as shown in FIG. 7, the device 38 may be inserted into the tibia 10 just under the lip of the cephalad portion 36 of the prosthesis.

Referring to FIG. 5, the device 38 is a generally elongate object with a surface 40 on which to support the prosthesis 34,36. The device 38 may be any size or shape deemed appropriate to support the joint replacement prosthesis 34,36 to be implanted in the patient and is desirably selected by the physician taking into account the morphology and geometry of the site to be treated. The physician is desirably able to select the desired size and/or shape of the device based upon prior analysis of the morphology of the target bone(s) using, for example, plain film x-ray, fluoroscopic x-ray, or MRI or CT scanning.

In a first illustrative embodiment, shown in FIG. 5, the device 38 has a screw-shaped configuration. The device 38 includes a tapered longitudinal body formed with external threads 42. The device 38 includes a head 40. The head 40 is adapted to engage the prosthesis. The head 40 may be formed with grooves 44 desirably configured to mate with an installation instrument, e.g., a screwdriver, to facilitate advancement and positioning of the device 38 in the bone.

The subsidence protection device 38 may be inserted into the talus 14 during installation of a total ankle joint replacement. As shown in FIG. 6A, the subsidence protection device may be inserted into the talus 14 just under the front lip of the prosthetic 34. It is also contemplated that in some situations it may be desirable for the subsidence protection device 38 to extend through the talus 14 into the calcaneous 16, as shown in FIG. 6B.

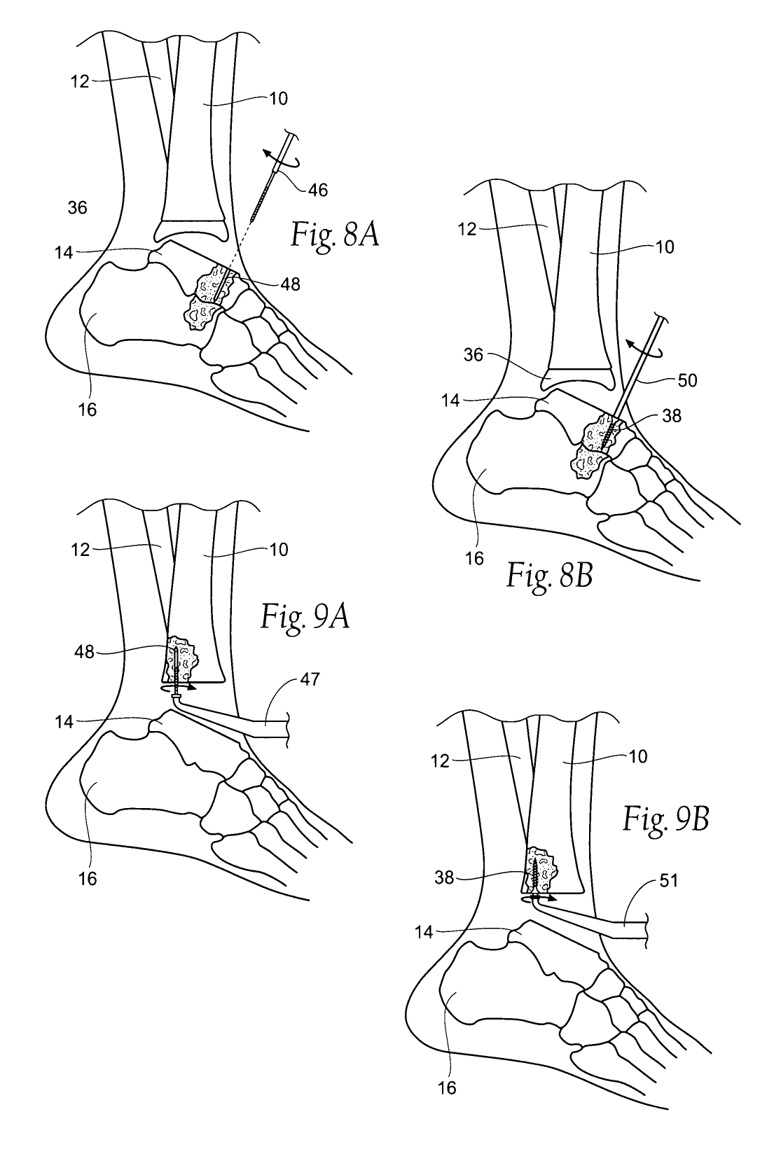

As shown in FIG. 8A, the physician may drill a bore 48 in the talus 14 using any appropriate surgical device, such as a standard surgical drill 46. In the illustrated embodiment, the drill bit is sized and configured to create a conical bore 48 similar in size and configuration to the device 38. The bore 48 is desirably sized and configured to permit tight engagement of the device 38 within the bore 48 and thereby restrict movement of the device 38 within the bore 48. The pre-formed bore 48 may be slightly smaller than the device 38, while still allowing the device 38 to be secured into position within the bore 48 by screwing. The drill bit is then withdrawn. Referring now to FIG. 8B, the device 38 is then screwed into the bore 48 using an appropriately sized and configured screwdriver 50.

It should also be understood that the subsidence protection device 38 may be inserted into the tibia 10 to protect subsidence of the cephalad portion 36 of the prosthesis into the tibia 10, as shown in FIGS. 9A and 9B. The subsidence protection device 38 may be inserted into the tibia 10 in the same manner as which the device 38 is inserted into the talus 14. First, the physician drills a bore 48 in the tibia 10, as shown in FIG. 9A. The bore 48 may be made using any appropriate surgical device, such as an appropriately sized and configured surgical drill 47. The bore 48 is desirably sized and configured to permit tight engagement of the device 38 within the bore 48 and thereby restrict movement of the device 38 within the bore 48. The bore 48 may be slightly smaller than the device 38, while still allowing the device 38 to be secured into position within the bore 48 by screwing. The drill bit is then withdrawn. The device 38 is then screwed into the bore 48 using an appropriately sized and configured screwdriver 51, as shown in FIG. 9B.

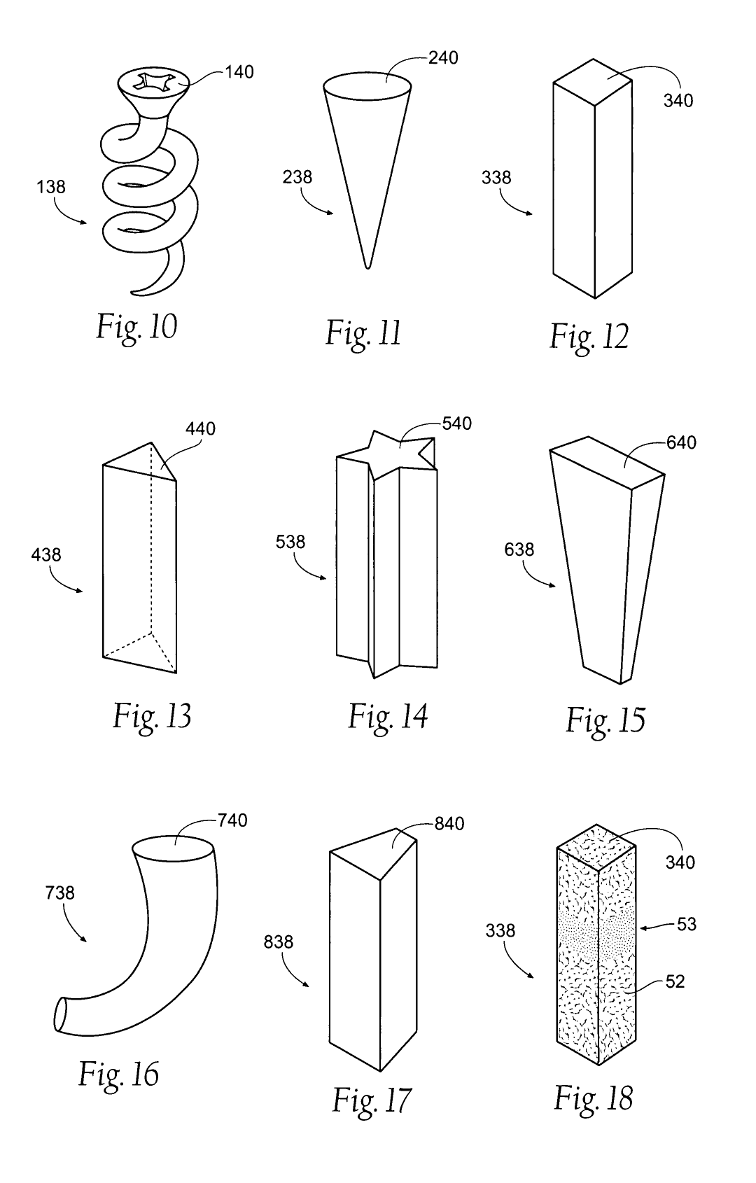

As stated above, the size and/or shape of the device 38 is selected to optimize support of the device to the ankle implant. The subsidence protector may be a corkscrew, as shown in FIG. 10 or a cone, as shown in FIG. 11. As shown in FIGS. 12 to 14, the subsidence protector may be an elongated body with a square, triangular, or star shaped cross section. The subsidence protector may be a wedge, as shown in FIG. 15, or a curved rod, as shown in FIG. 16. The subsidence protector may be an elongated body with a trapezoidal cross section, as shown in FIG. 17. The preceding configurations are given by way of example. It should be understood that the subsidence protector may be of any configuration suitable to support the prosthesis.

The alternative embodiment described above and shown in FIG. 10 may be screwed into a preformed bore in either the talus or tibia in the same manner as described above. The alternate embodiment described above and shown in FIG. 11 may be tapped into a pre-formed bore in either the talus or tibia. The bore in the talus or tibia may be formed as described above.

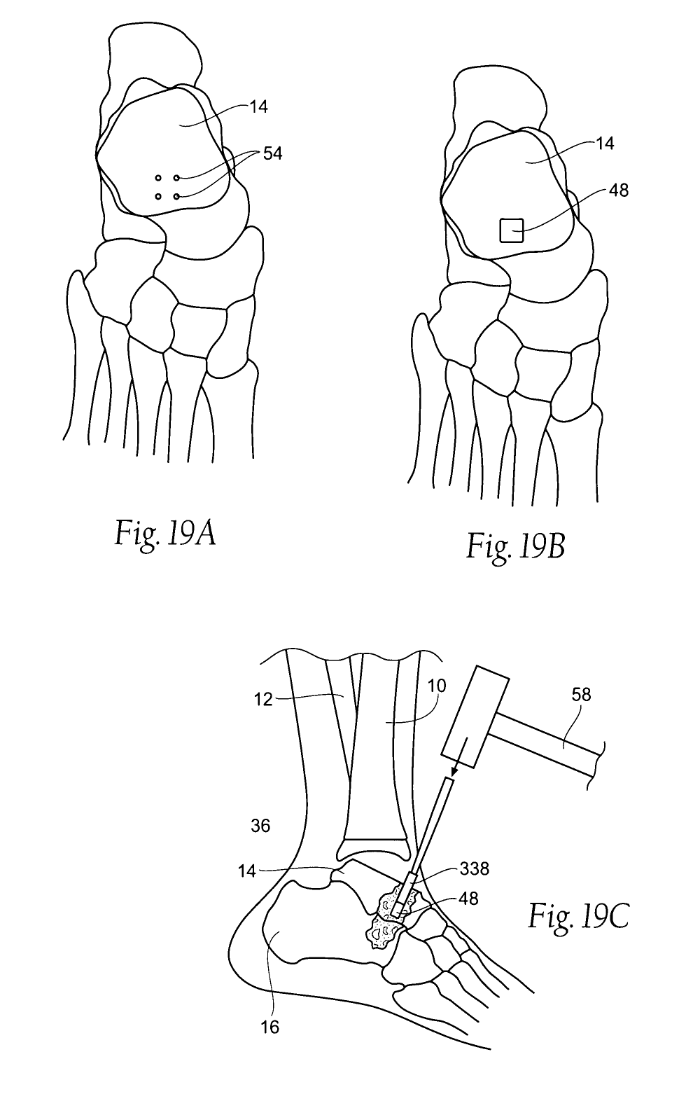

The alternative embodiment 338 shown in FIG. 12 may be inserted by tapping the device into a pre-formed bore, as shown in FIGS. 19A to 19C. At least one pilot hole 54 is first drilled into the bone at the insertion site using conventional surgical techniques, as shown in FIG. 19A. In the embodiment shown in FIG. 19A, four pilot holes are drilled in order to create a square bore 48. However, it is to be understood that the number and configuration of the pilot holes 54 may vary as necessary or as desired. The physician can then saw, using conventional methods such as a surgical saw, between and around the pilot holes 54 to prepare a bore 48 appropriately sized and configured to receive the device 338, as shown in FIG. 19B. The bore 48 is desirably sized and configured to permit tight engagement of the device 338 within the bore 48 and thereby restrict movement of the device 338 within the bore. The pre-formed bore 48 may be slightly smaller than the device 238, while still allowing the device 338 to be secured into position within the bore 48 by tapping using an appropriately sized and configured mallet 58, as shown in FIG. 19C. The embodiments shown in FIGS. 13 to 17 are inserted in the same manner as described in FIGS. 19A to 19C, however, as will be clear to one of skill in the art, the shape and size of the bore 48 will vary with the particular configuration of the device.

The device 38 may be made of various materials commonly used in the prosthetic arts including, but not limited to, metals, ceramics, tantalum, polyethylene, biologic type polymers, hydroxyapetite, rubber, titanium, titanium alloys, tantalum, chrome cobalt, surgical steel, or any other total joint replacement metal and/or ceramic, bony in-growth surface, sintered glass, artificial bone, any porous metal coat, metal meshes and trabeculations, metal screens, uncemented metal or ceramic surface, other bio-compatible materials, or any combination thereof.

It may be desirable to provide surface texturing 52 along at least a portion of the length of the device 338 to promote bony in-growth on its surface, an example of which is shown in FIG. 18. Although the surface texturing is only shown with respect to one particular embodiment 338, it should be understood that the surface texturing could be applied to the device 38 regardless of the specific configuration of the device 38. The surface texturing 52 can comprise, e.g., through holes, and/or various surface patterns, and/or various surface textures, and/or pores, or combinations thereof. The device 338 can be coated or wrapped or surfaced treated to provide the surface texturing, or it can be formed from a material that itself inherently possesses a surface conducing to bony in-growth, such as a porous mesh, hydroxyapetite, or other porous surface.

It may further be desirably for the device 38 to be covered with various coatings 53 such as antimicrobial, antithrombogenic, and osteoinductive agents, or a combination thereof. An example of such a coating 53 on the device 338 is shown in FIG. 18. Although the surface texturing is only shown with respect to one particular embodiment 338, it should be understood that the surface texturing could be applied to the device 38 regardless of the specific configuration of the device 38.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

I/We claim:

1. A subsidence protection device comprising
an elongate body sized and configured for insertion into a bone, said body including a head sized and configured to engage at least a portion of a prosthetic inserted into said bone.

2. A device according to claim 1 wherein said elongate body is tapered.

3. A device according to claim 2 wherein said elongate body is formed with external threads.

4. A device according to claim 3 wherein said head is configured to engage a screwdriver.

5. A device according to claim 1 wherein at least a portion of the body includes a region permitting bony in-growth and/or through-growth.

6. A device according to claim 1 wherein the body comprises a prosthetic material.

7. A device according to claim 1 wherein the body comprises a biological material.

8. A device according to claim 1 wherein said body is corkscrew-shaped.

9. A device according to claim 1 wherein said body is cone-shaped.

10. A device according to claim 1 wherein said body has a square cross section.

11. A device according to claim 1 wherein said body has a triangular cross section.

12. A device according to claim 1 wherein said body has a star-shaped cross section.

13. A device according to claim 1 wherein said body is wedge-shaped.

14. A device according to claim 1 wherein said body comprises a curved rod.

15. A device according to claim 1 wherein said body has a trapezoidal cross section.

16. A method comprising
providing a subsidence protection device comprising a body and a head;
selecting a bone site;
forming a cavity in the bone site sized and configured to receive the body; and
inserting the body in the bone cavity.

17. The method according to claim 16 wherein said body is formed with external threads.

18. The method according to claim 17
wherein the body is screwed into the bone cavity.

19. The method according to claim 16
wherein the body is inserted in the bone cavity by tapping.

20. The method according to claim 16 wherein the selected bone site is in the tibia.

21. The method according to claim 16 wherein the selected bone site is in the talus.
